(19) United States
(12) Patent Application Publication
(10) Pub. No.: US 2001/0020685 A1
(43) Pub. Date: Sep. 13, 2001

(54) Detector and method for detecting plate-shaped or sheet-shaped body

(75) Inventor: Tomohiko Serita, Tokyo (JP)

Correspondence Address: MORGAN, LEWIS & BOCKIUS, 1800 M STREET NW, WASHINGTON, DC, 20036-5869, US

(73) Assignee: MAX CO., LTD.

(21) Appl. No.: 09/801,785

(22) Filed: Mar. 9, 2001

(30) Foreign Application Priority Data
Mar. 9, 2000 (JP) P2000-65152

(51) Int. Cl. A61N 5/00; G21G 5/00

(52) U.S. Cl. 250/492.1

(57) ABSTRACT

A method of detecting a plate-shaped or sheet-shaped body includes the steps of: arranging a first optical sensor in parallel with one face of a body; arranging a second optical sensor in parallel with the other face of the body and also in parallel with the first optical sensor; turning on a light emitting section of the first optical sensor and a light emitting section of the second optical sensor alternately; monitoring the fluctuation of a detection level detected by a light receiving section of the first optical sensor and the fluctuation of a detection level detected by a light receiving section of the second optical sensor; and recognizing the body when the fluctuation of the detection level exceeds a predetermined value.

### BACKGROUND OF THE INVENTION

1. Field of the Invention

The present invention relates to a detector and method for detecting a plate-shaped or sheet-shaped body with optical sensors.

2. Description of the Related Art

Conventionally, as shown in FIG. 5(a), when existence of a plate-shaped or sheet-shaped body is detected with an optical sensor, a light emitting section 10 and a light receiving section 11 are arranged perpendicular to a body A. The existence of the plate-shaped or sheet-shaped body is judged by a change in a detection level of light detection conducted by the light receiving section 11 when a ray of light incident upon the light receiving section 11 is shut off by the body A.

However, in the case where the optical sensor can not be arranged in the direction perpendicular to the body A, as shown in FIG. 5(b), it is possible to adopt a method in which the light emitting section 12 and the light receiving section 13 are arranged on one face side of the body A so that reflected light on the reflecting plate 14 can be detected. However, in the case where there are no spaces for arranging the optical sensor and reflecting plate on either sides of the body A, it is difficult to detect the plate-shaped or sheet-shaped body by the optical sensor.

### SUMMARY OF THE INVENTION

It is a task of the present invention to solve the above problems, that is, it is a task of the present invention to provide a detector and method for detecting a plate-shaped or sheet-shaped body with an optical sensor capable of detecting the plate-shaped or sheet-shaped body from a position except for both sides of the body.

In order to solve the above problems, the present invention provides a method of detecting a plate-shaped or sheet-shaped body with an optical sensor composed of a light emitting section and a light receiving section opposed to each other at a predetermined interval, including the steps of: arranging a first optical sensor in parallel with one face of the body; arranging a second optical sensor in parallel with the other face of the body and also in parallel with the first optical sensor; turning on a light emitting section of the first optical sensor and a light emitting section of the second optical sensor alternately; monitoring a fluctuation of a detection level detected by the light receiving section of the first optical sensor and a fluctuation of a detection level detected by the light receiving section of the second optical sensor; and recognizing a body when the fluctuation of the detection level exceeds a predetermined value.

### BRIEF DESCRIPTION OF THE DRAWINGS

FIGS. 1(a) and 1(b) are respectively a front view and a plan view showing a relation between a body and an optical sensor relating to the present invention.

FIG. 2 is a schematic illustration of a drive circuit of an optical sensor.

FIGS. 3(a), 3(b), 3(c) and 3(d) are front views for explaining a relation between an optical sensor and a body.

FIG. 4 is a time chart for explaining operation of an optical sensor.

FIGS. 5(a) and 5(b) are views showing a conventional detection method of detecting a body by an optical sensor.

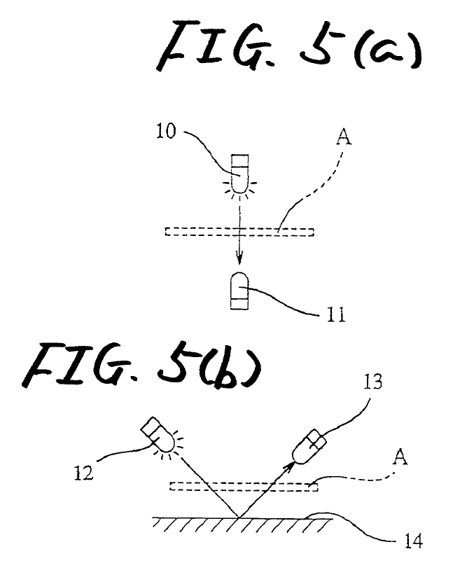

### DETAILED DESCRIPTION OF THE PREFERRED EMBODIMENT

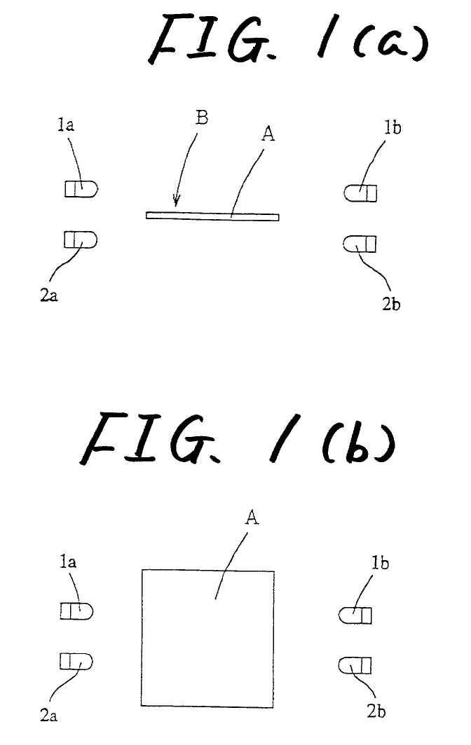

FIGS. 1(a) and 1(b) are views showing a detecting method of detecting a plate-shaped or sheet-shaped body A with an optical sensor. In this method, on both sides of the body A (in an upper portion and a lower portion of the body A in FIG. 1(a)), there are provided a first optical sensor 1 and second optical sensor 2. The optical sensors 1, 2 are composed in such a manner that light emitting sections (light emitting diodes) 1a, 2a and light receiving sections (photo-transistors) 1b, 2b are respectively arranged being opposed to each other. Light emitted by the light emitting section 1a (referred to as a first light emitting section hereinafter) of the first optical sensor 1, which is arranged on one face side (upper side in FIG. 1(a)) of the body A, is received by the light receiving section 1b (referred to as a first light receiving section hereinafter) of the first optical sensor 1 and also received by the light receiving section 2b (referred to as a second light receiving section hereinafter) of the second light emitting section 2. Light emitted by the light emitting section 2a (referred to as a second light emitting section hereinafter) of the second optical sensor 2 is received by the second light receiving section 2b and the first light receiving section 1b. Output voltages of the first light receiving section 1b and the second light receiving section 2b are respectively monitored. Therefore, it is possible to detect whether or not the body A exists in a measurement stage by the fluctuation of the detection level.

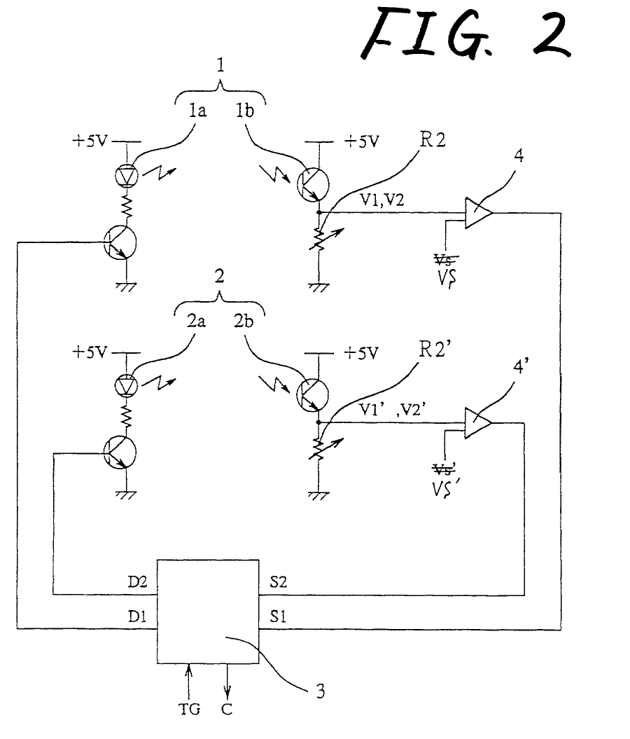

FIG. 2 is a view showing an example of the optical sensor control circuit. The first light emitting section 1a and the second light emitting section 2a are alternately turned on by drive signals D1, D2 which are alternately outputted from the control section 3. Therefore, the first light receiving section 1b and the second light receiving section 2b do not simultaneously receive the light emitted from the two light emitting sections. Output levels of the light receiving sections are compared with judgment reference values VS, VS′ in the comparative circuits 4, 4′. When the output levels are lower than the judgment reference values, detection signals S1, S2 are inputted into the control section, so that control signal C can be outputted from the control section 3.

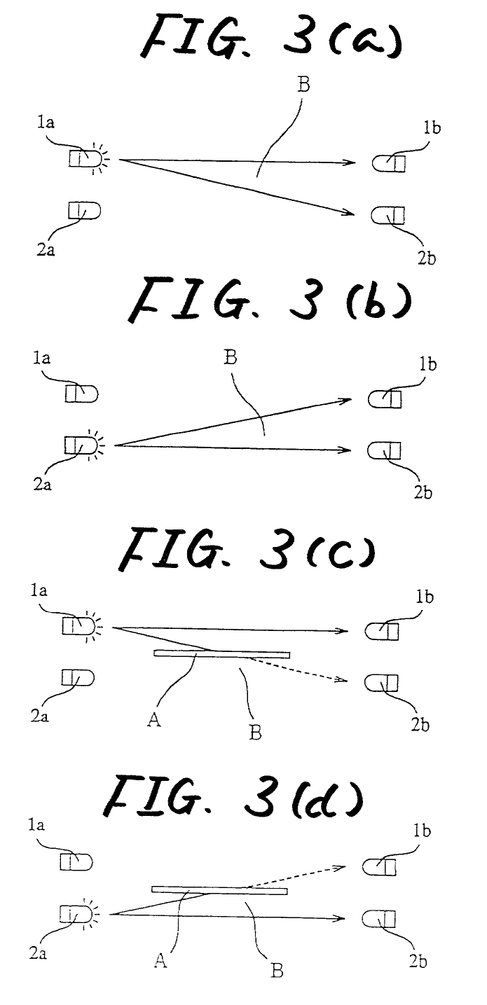

As shown in FIG. 3(a), variable resistor R2′ is adjusted so that the output voltages of the first light receiving section 1b and the second light receiving section 2b can respectively become predetermined values of V1 and V1′ (V1: 5 V, V1′: 3.8 V in the present invention) when the first light emitting section 1a is turned on under the condition of no-load (the body A is not in a measurement stage B). Further, as shown in FIG. 3(b), variable resistor R2 is adjusted so that the output voltages of the first light receiving section 1b and the second light receiving section 2b can respectively become predetermined values of V2 and V2′ (V2: 3.8 V, V2′: 5 V in the present invention) when the second light emitting section 2a is turned on.

Since the first light receiving section 1b is opposed to the first light emitting section 1a under the condition of no-load, the collector current is saturated. Therefore, the output voltage V1 becomes an impressed voltage (5 V). Since the second light receiving section 2b is opposed to the second light emitting section 2a under the condition of no-load, the collector current is saturated. Therefore, output voltage V2′ becomes an impressed voltage (5 V). However, since light is obliquely incident upon the light receiving section which is located diagonally with respect to the light emitting section that has been turned on, a quantity of light is reduced, and the collector current is not saturated. Therefore, it is possible to adjust the respective output voltages to be 3.8 V by variable resistors R2 and R2′.

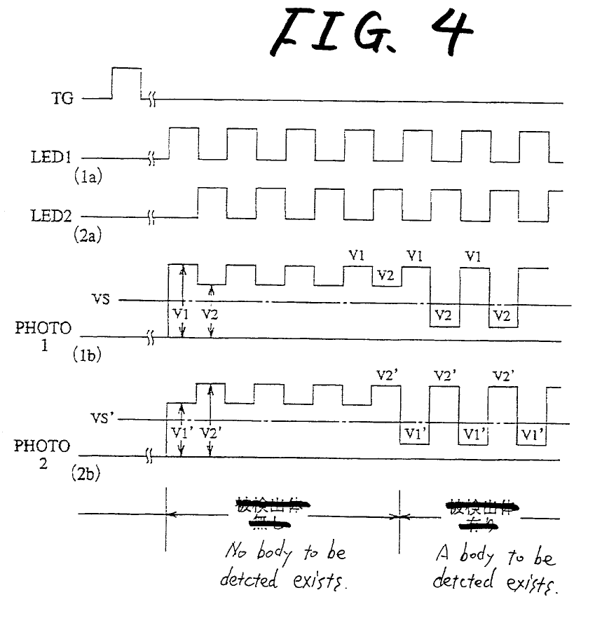

The control section 3 starts detecting the body A at predetermined timing TG, and the first light emitting section 1a and the second light emitting section 2a are alternately turned on. Therefore, as shown in the time chart of FIG. 4, under the condition of no-load (the body A is not on a measurement table B), V1, V2 and V1′, V2′ of the output voltages with respect to the first light receiving section 1b and the second light receiving section 2b are alternately outputted.

When the body A exists on the measurement table B, as shown in FIG. 3(c), light emitted from the first light emitting section 1a to the second light receiving section 2b is shut off by the body A, so that output voltage V1′ is lowered. Also, when the body A exists on measurement table B. as shown in FIG. 3(d), light emitted from the second light emitting section 2a to the first light receiving section 1b is shut off by the body A, so that output voltage V2 is lowered. In the case where the fluctuation of output voltage V1′ and that of output voltage V2 exceed a predetermined value, which is set at 0.5 V in the present invention, that is, in the case where the fluctuation of output voltage V1′ and that of output voltage V2 are lower than judgment reference values VS, VS′, detection signals S1, S2 are inputted into the control section 3, so that the control section can judge that the body A exists on the measurement table B.

As described above, in the case where the body A does not exist on the measurement table B, output voltages of the light receiving sections keep the predetermined values V1, V2, V1′, V2′. However, in the case where the body A exists on the measurement table B, light emitted from the first light emitting section 1a is shut off by the body A, so that output voltage V1′ of the second light receiving section 2b becomes lower than the output voltage in a steady state, and light emitted from the second light emitting section 2a is shut off by body A, so that output voltage V2 of the first light receiving section 1b becomes lower than the output voltage in a steady state.

In the present invention, when the fluctuation exceeds a predetermined value (0.5 V in the present invention), that is, in the case where V2 is not higher than 3.3 V, and V1′ is not higher than 3.3 V, it is judged that the body A has been recognized. Therefore, the control section 3 outputs a control signal C so as to inform the recognition of the body A with an information means such as an LED or speaker not shown. In the case of a system in which the body A is not allowed to exist on measurement table B, the system is stopped until body A is removed from measurement table B.

According to the present invention, even when there is provided no space to arrange an optical sensor on either sides of a plate-shaped or sheet-shaped flat body, as long as there is provided a space in which light can be made to pass on both sides of the body, the flat body can be detected by an optical sensor. Therefore, the present invention can provide a detection method effectively used when the device is incorporated into a machine.

While only a certain embodiment of the invention has been specifically described herein, it will be apparent that numerous modifications may be made thereto without departing from the spirit and scope of the invention.

What is claimed is:

1. A method of detecting a plate-shaped or sheet-shaped body with an optical sensor including a light emitting section and a light receiving section opposed to each other at a predetermined interval, comprising the steps of:
arranging a first optical sensor in parallel with one face of the body;
arranging a second optical sensor in parallel with the other face of the body and in parallel with the first optical sensor;
turning on a first light emitting section of the first optical sensor and a second light emitting section of the second optical sensor alternately;
monitoring a first fluctuation of a detection level detected by a first light receiving section of the first optical sensor and a second fluctuation of a detection level detected by a second light receiving section of the second optical sensor; and
recognizing the body when one of the first and second fluctuations exceeds a predetermined value.

2. A detector for a plate-shaped or sheet-shaped body comprising:
a first optical sensor including a first light emitting section and a first light receiving section opposed to each other at a first interval, said first optical sensor arranged in parallel with one face of the body and;
a second optical sensor including a second light emitting section and a second light receiving section opposed to each other at a second interval, said second optical sensor arranged in parallel with the other face of the body and in parallel with said first optical sensor;
a first comparative circuit for comparing a first output level from the first light receiving section with a first judgment reference value and for outputting a first detection signal when the first output level is lower than the first judgment reference value;
a second comparative circuit for comparing a second output level from the second light receiving section with a second judgment reference value and for outputting a second detection signal when the second output level is lower than the second judgment reference value;
a control section for alternately outputting a first drive signal to the first light emitting section and a second drive signal to the second light emitting section so that the first and second light emitting sections are alternately turned on and off, and for outputting a control signal when said control section receives one of the first and second detection signals.
